(19) United States
(12) Patent Application Publication
(10) Pub. No.: US 2006/0111407 A1
(43) Pub. Date: May 25, 2006

(54) Association between a heterocyclic compound stimulating lipid and carbohydrate metabolisms and an antioxidant agent for treating obesity

(76) Inventors: Louis Casteilla, Escalquens (FR); Luc Penicaud, Toulouse (FR); Pascal Rerthelot, Haubourdin (FR); Catherine Dacquet, Paris (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 10/530,945

(22) PCT Filed: Oct. 10, 2003
(86) PCT No.: PCT/FR03/02987

§ 371 Date: Apr. 8, 2005

(30) Foreign Application Priority Data
Oct. 11, 2002 (FR) 02/12648

(51) Int. Cl. A61K 31/428; A61K 31/423; A61K 31/404

(52) U.S. Cl. 514/367; 514/375; 514/418; 548/157; 548/221; 548/484

(57) ABSTRACT

The invention relates to a combination comprising a compound favouring lipid and carbohydrate metabolisms and an antioxidant agent and to pharmaceutical compositioins comprising the combination.

The present invention relates to a new association between a heterocyclic compound and an antioxidant agent for obtaining pharmaceutical compositions for use in the treatment and/or prevention of obesity and of overweight characterised by a body mass index greater than 25.

Obesity is a major public health problem in all developed countries. It is also increasing steadily in developing countries and is affecting an ever younger population. Obesity is a well-established risk factor for cardiovascular diseases and is associated with a significantly increased risk of cerebro-vascular accidents, non-insulin-dependent diabetes, vesicular calculi, respiratory dysfunction, osteoarthritis, several forms of cancer and premature death.

It has been found that, in obese people, the generation of reactive oxygenated species released by monocytes and leukocytes is greatly increased with respect to non-obese subjects (J. Clin. Endocrinol. Metab., 2001, 86, 355-362). Elevated plasma concentrations of alpha tumour necrosis factor (TNFα) in obese people stimulate inflammatory processes (J. Clin. Endocrinol. Metab., 1998, 83, 2907-2910) and are responsible for the generation of reactive oxygenated species by leukocytes (Oncogene, 1998, 17, 1639-1651).

The pathological state of obesity is also associated with increased oxidation of lipids and proteins, which may be the cause of high plasma levels of 9- and β-hydroxy-octadecadienoic acids (9-HODE and 13-HODE) (Totowa: Humano. Press., 1998, 147-155), key indices of lipid peroxidation (J. Clin. Endocrinol. Metab., 2001, 86, 355-362). In parallel, the “antioxidant” capabilities of the body are reduced.

In obese subjects, it has been shown that excessive food intake causes major lipid and protein damage. Over-consumption of calories by obese people can cause the formation of free radicals and expose them to significant oxidative lesions which help to maintain the state of obesity.

The specific markers of oxidation are significantly reduced by a 48-hour fast or by calorie restriction accompanying weight loss (J. Clin. Endocrinol. Metab., 2001, 86, 355-362).

A strategy aimed at reducing the “oxidative burden” on the body by favouring the lipid and carbohydrate metabolisms should result in an exacerbation of the effects and, as a consequence, in weight loss in obese or overweight subjects.

The present invention relates more specifically to the association between a compound favouring the lipid and carbohydrate metabolisms of the body and an antioxidant agent.

This association is novel and exhibits pharmacological properties that are entirely surprising in the area of obesity.

More specifically, the invention relates to the association between a compound favouring the lipid and carbohydrate metabolisms which has a heterocyclic structure and an antioxidant agent.

The heterocyclic compounds favouring the lipid and carbohydrate metabolisms in accordance with the invention are, more specifically, compounds of formula (I) described in the Patent Application WO 01/57002: 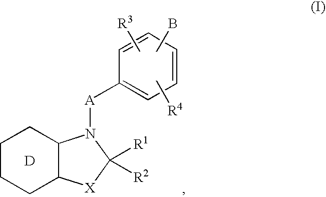
wherein:
X represents an oxygen or sulphur atom or a CH₂ or 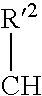
   group (wherein
R² together with R² forms an additional bond),
R¹ and R², which may be identical or different, each represents a hydrogen atom, a linear or branched (C₁-C₆)alkyl group, an aryl group, an aryl-(C₁-C₆)alkyl group in which the alkyl moiety may be linear or branched, an aryloxy group, an aryl-(C₁-C₆)alkoxy group in which the alkoxy moiety may be linear or branched, a linear or branched (C₁-C₆)alkoxy group, a hydroxy group, an amino group, a linear or branched (C₁-C₆)alkylamino group or a dialkylamino group in which the alkyl moieties are linear or branched C₁-C₆,
or R¹ and R² together form an oxo, thioxo or imino group,
it being possible furthermore for R² to form with R¹² an additional bond,
A represents a (C₁-C₆)alkylene chain in which a CH₂ group may be replaced by a hetero atom selected from oxygen and sulphur, by an NRₐ group (wherein Rₐ represents a hydrogen atom or a linear or branched (C₁-C₆)alkyl group), or by a phenylene or naphthylene group,
B represents a linear or branched (C₁-C₆)alkyl group or a linear or branched (C₂-C₆)alkenyl group, those groups being substituted by a R⁵ group,
by a group of formula (II): 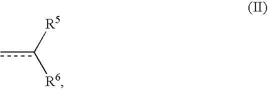
or by a group of formula (III): 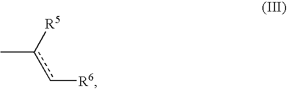
in which groups:
   the representation [symbol] denotes that the bond is single or double,
   R⁵ represents a 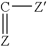
   group wherein Z represents a sulphur atom or an oxygen atom and Z′ represents an OR or NRR′ group,
   and R⁶ represents a group 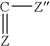
   wherein Z″ represents a Z′ or R group,
   (wherein R and R′, which may be identical or different, each represents a R″ or —C(Me)₂COOR″ group wherein R″ represents a hydrogen atom or a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched, or a linear or branched (C₁-C₆)polyhaloalkyl group),
R³ and R⁴, which may be identical or different, each represents a hydrogen atom, a halogen atom or a R, OR or NRR′ group (wherein R and R′ are as defined hereinbefore),
or R³ and R⁴ together with the carbon atoms carrying them, when they are carried by two adjacent carbon atoms, form a ring that has 5 or 6 ring members and that may contain a hetero atom selected from oxygen, sulphur and nitrogen,
D represents:
a benzene nucleus, in which case X cannot represent a group 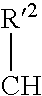
as defined hereinbefore,
or D represents a pyridine, pyrazine, pyrimidine or pyridazine nucleus, those five nuclei being unsubstituted or substituted by from 1 to 3 identical or different groups selected from R, OR, S(O)ₙR, 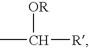
C(Z)OR, NRR′, C(Z)NRR′, 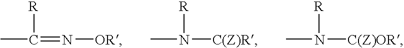
(in which groups R, R′ and Z are as defined hereinbefore and n is 0, 1 or 2), cyano, nitro and halogen atoms,
   wherein:
when A represents a CH₂ group, B cannot represent a linear or branched (C₁-C₆)alkyl group substituted by a group 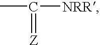
when the groups A and B are in the ortho position in relation to one another on the benzene nucleus carrying them, B cannot represent a linear or branched (C₂-C₆)— alkenylene group substituted by a group 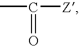
when A represents a group 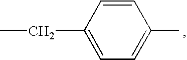
   B cannot represent a —CH₂—COOH group,
aryl is to be understood as a phenyl, naphthyl or biphenyl group, which groups may be partially hydrogenated,
heteroaryl is to be understood as any mono- or bi-cyclic aromatic group containing from 5 to 10 ring members, which may be partially hydrogenated on one of the rings in the case of bicyclic heteroaryls, and which contains from 1 to 3 hetero atoms selected from oxygen, nitrogen and sulphur,
   wherein the aryl and heteroaryl groups so defined may be substituted by from 1 to 3 groups selected from linear or branched (C₁-C₆)alkyl, linear or branched (C₁-C₆)alkoxy, carboxy, formyl, NR_{b}R_{c} (wherein R_{b} and R_{c}, which may be identical or different, each represents a hydrogen atom, a linear or branched (C₁-C₆)alkyl group, an aryl group or a heteroaryl group), ester, amido, nitro, cyano, O—C(Me)₂COOR″ (wherein R″ is as defined hereinbefore) and halogen atoms,
   their enantiomers and diastereoisomers, and also addition salts thereof with a pharmaceutically acceptable acid or base.

Even more preferably, the heterocyclic compounds of the association according to the invention are:
dimethyl 2-{4-[2-(6-benzoyl-2-oxo-1,3-benzothiazol-3(2H)-yl)ethoxy]benzyl}-malonate,
3-methoxy-2-{4-[2-(6-[(methoxyimino)(phenyl)methyl]-2-oxo-1,3-benzo-thiazol-3(2H)-yl)ethoxy]benzyl}-3-oxopropanoic acid,
dimethyl 2-{4-[2-(6-[(hydroxyimino)(phenyl)methyl)-2-oxo-1,3-benzothiazol-3(2H)-yl)ethoxy]benzyl}malonate,
2-{4-[2-(6-benzoyl-2-oxo-1,3-benzothiazol-3(2H)-yl)ethoxy]benzyl}-3-methoxy-3-oxopropanoic acid,
dimethyl 2-{4-[2-(6-[2-chlorophenyl)(methoxyimino)methyl]-2-oxo-1,3-benzo-thiazol-3(2H)-yl-ethoxy]benzylidene}malonate,
dimethyl 2-{4-[2-(6-[(3-chlorophenyl)(methoxyimino)methyl]-2-oxo-1,3-benzo-thiazol-3 (2H)-yl)ethoxy]benzyl}malonate,
dimethyl 2-{4-[2-(6-[(1,1′-biphenyl]-4-yl(methoxyimino)methyl]-2-oxo-1,3-benzothiazol-3(2H)-yl)ethoxy]benzyl}malonate,
methyl 3-{4-[2-(6-[(methoxyimino)(phenyl)methyl]-2-oxo-1,3-benzothiazol-3(2H)-ethoxy]phenyl}propanoate,
methyl 2-{4-[2-(6-benzoyl-2-oxo-1,3-benzothiazol-3(2H)-yl)ethoxy]benzyl}-3-(methylamino)-3-oxopropanoate,
ethyl 2-benzoyl-3-{4-[2-(6-benzoyl-2-oxo-1,3-benzothiazol-3 (2H)-yl)ethoxy]-phenyl}-2-propenoate,
2-{4-[2-(6-benzoyl-2-oxo-1,3-benzothiazol-3(2H)-yl)ethoxy]benzyl}-3-tert-butoxy-3-oxopropanoic acid,
methyl 2-{4-[2-(6-[(methoxyimino)(phenyl)methyl]-2-oxo-1,3-benzothiazol-3 (2H)-ethoxy]benzyl}-3-(methylamino)-3-oxopropanoate,
methyl 2-{4-[2-(6-[(methoxyimino)(phenyl)methyl]-2-oxo-1,3-benzothiazol-3(2H)-yl)ethoxy]benzyl}-3-oxo-phenylpropanoate,
2-{4-[2-(6-[(hydroxyimino)(phenyl)methyl]-2-oxo-1,3-benzothiazol-3(2H)-yl)ethoxy]benzyl}-3-methoxy-3-oxopropanoic acid,
3-tert-butoxy-2-{4-[2-(6-[(methoxyimino)(phenyl)methyl]-2-oxo-1,3-benzo-thiazol-3(2H)-yl)ethoxy]benzyl}-3-oxopropanoic acid,
3-tert-butoxy-2-{4-[2-(6-[(hydroxyimino)(phenyl)methyl]-2-oxo-1,3-benzo-thiazol-3(2H)-yl)ethoxy]benzyl}-3-oxopropanoic acid,
3-isopropoxy-2-{4-[2-(6-[(methoxyimino)(phenyl)methyl]-2-oxo-1,3-benzo-thiazol-3(2H)-yl)ethoxy]benzyl}-3-oxopropanoic acid,
2-{4-[2-(6-[(hydroxyimino)(phenyl)methyl]-2-oxo-1,3-benzothiazol-3(2H)-yl)ethoxy]benzyl}-3-isopropoxy-3-oxopropanoic acid,
3-butoxy-2-{4-[2-(6-[(methoxyimino)(phenyl)methyl]-2-oxo-1,3-benzothiazol-3(2H)-yl)ethoxy]benzyl}-3-oxopropanoic acid,
dimethyl 2-{4-[2-(6-(3-chlorobenzoyl)-2-oxo-1,3-benzothiazol-3(2H)-yl)ethoxy]-benzyl}malonate,
dimethyl 2-{4-[2-(6-[(methoxyimino)(phenyl)methyl]-2-oxo-1,3-benzothiazol-3 (2H)-yl)ethoxy]benzyl}malonate,
   their enantiomers and diastereoisomers, and also addition salts thereof with a pharmaceutically acceptable acid or base.

Antioxidant agents according to the invention are, more specifically, anti-free radical agents or free-radical trapping agents, antilipoperoxidant agents, chelating agents or agents capable of regenerating endogenous antioxidants such as glutathione, vitamin C or vitamin E, and also addition salts thereof with a pharmaceutically acceptable acid or base.

Amongst the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid, etc.

Amongst the pharmaceutically acceptable bases there may be mentioned, without implying any limitation, sodium hydroxide, potassium hydroxide, triethylamine, tert-butylamine, etc.

The antioxidant agent of the association according to the invention is more preferably represented by quinone compounds such as ubiquinone or coenzyme Q₁₀, which acts as a free-radical trapping agent but which is also capable of regenerating vitamin E.

The association to which preference is given in accordance with the invention is dimethyl 2-{4-[(6-benzoyl-2-oxo-1,3-benzothiazol-3(2H)-yl)ethoxy]benzyl}malonate and coenzyme Q₁₀.

Furthermore, the association according to the invention between a compound favouring the lipid and carbohydrate metabolisms and an antioxidant agent has entirely surprising pharmacological properties: the Applicant has discovered that a synergy exists between the two compounds of the association allowing a very significant reduction in body fat to be obtained, making it of use in the treatment and/or prevention of obesity and of overweight characterised by a body mass index greater than 25.

In the United States, obesity affects 20% of men and 25% of women. Patients having a body mass index (BMI=weight (kg)/height² (m²)) greater than or equal to 30 are considered to be obese (Int. J. Obes., 1998, 22, 39-47; Obesity Lancet, 1997, 350, 423-426). Obesity (BMI≧30) and overweight (25<BMI<30) can have various origins: they may come about following deregulation of food intake, following hormonal disturbance, or following administration of a treatment: treating type II diabetes with sulphonylureas causes patients to gain weight. Similarly, in type I (insulin-dependent) diabetes, insulin therapy is also a cause of weight gain in patients (In Progress in Obesity Research, 8^{th} International Congress on Obesity, 1999, 739-746; Annals of Internal Medicine, 1998, 128, 165-175).

Obesity and overweight are well-established risk factors for cardiovascular diseases: they are associated with a significant increase in the risk of cerebro-vascular accidents and non-insulin-dependent diabetes, because they predispose to insulin-resistance, dyslipidaemia and the appearance of macrovascular disorders (nephropathy, retinopathy, angiopathy).

Further pathologies are the consequence of obesity or overweight: there may be mentioned, in particular, vesicular calculi, respiratory dysfunction, several forms of cancer and, in the case of very severe obesity, premature death (N. Engl. J. Med., 1995, 333, 677-385; JAMA, 1993, 270, 2207-2212).

The association according to the invention allows a weight loss to be obtained which, even if moderate, significantly reduces all the risk factors associated with obesity (Int. J. Obes., 1997, 21, 55-9; Int. J. Obes., 1992, 21, S5-9).

The association according to the invention will therefore be found to be useful in the treatment and/or prevention of obesity and of overweight characterised by a body mass index greater than 25.

The invention accordingly relates to the use of the association between a compound favouring the lipid and carbohydrate metabolisms and an antioxidant agent in obtaining pharmaceutical compositions intended for the treatment and/or prevention of obesity and of overweight characterised by a body mass index greater than 25 and less than 30.

In particular, the association according to the invention is of use in the treatment and/or prevention of obesity and of overweight characterised by a body mass index greater than 25 and less than 30 caused by a therapeutic treatment, such as treatment for type I or II diabetes.

The invention accordingly relates to the use of the association between a compound favouring the lipid and carbohydrate metabolisms and an antioxidant agent in obtaining pharmaceutical compositions intended for the treatment and/or prevention of obesity and of overweight characterised by a body mass index greater than 25 and less than 30 caused by a therapeutic treatment, such as treatment for type I or II diabetes.

The invention relates also to pharmaceutical compositions comprising the association between a compound favouring the lipid and carbohydrate metabolisms and an antioxidant agent, as defined hereinbefore, in combination with one or more pharmaceutically acceptable excipients.

Among the pharmaceutical compositions according to the invention, there may be mentioned, more especially, those that are suitable for oral, parenteral or nasal administration, tablets or dragées, sublingual tablets, gelatin capsules, lozenges, suppositories, creams, ointments, dermal gels, etc.

In particular, the invention relates to pharmaceutical compositions comprising a compound of formula (I) as defined hereinbefore and an antioxidant agent such as coenzyme Q₁₀ or vitamin E, in combination with one or more pharmaceutically acceptable excipients.

The dosage used varies according to the sex, age and weight of the patient, the administration route, the nature of the therapeutic indication or of any associated treatments and ranges from 0.1 mg to 1 g of each component of the association per 24 hours in one or more administrations.

The following Examples illustrate the invention but do not limit it in any way.

### EXAMPLE A

### Change in Body Weight

Male C57 Black 6 ob/ob mice from 8 to 12 weeks old were used. After being placed in quarantine for one week, they were weighed and then randomised as a function of their weight and 6 homogeneous groups (starting weights not significantly different) were formed. After being weighed, the various compounds under test were injected by the intraperitoneal route once a day for 7 days. The compounds were injected in a solution of DMSO 5%/Solutol 15%/qsp H₂O heated at 65° C. to ensure good dissolution. In addition, the solution was pre-heated before injection. The mice were weighed every day and the weight obtained after 7 days of treatment was recorded.

The results obtained with the association dimethyl 2-{4-[2-(6-benzoyl-2-oxo-1,3-benzoylthiazol-3(2H)-yl)ethoxy]benzyl}malonate (compound A)+coenzyme Q₁₀ and dimethyl 2-{4-[2-(6-benzoyl-2-oxo-1,3-benzoylthiazol-3(2H)-yl)ethoxy]benzyl}malonate (compound A)+vitamin E are indicated below and are expressed as the percentage weight change with respect to the control (corresponding to mice treated with the injection solvent for 7 days):

[image]

The results obtained clearly show:

that the association enables the weight of obese mice to be reduced significantly,
that there exists a synergy between the 2 components of the association, the weight loss found being much greater in the case of the association than in the case of each component administered on its own.

### EXAMPLE B

### Pharmaceutical Composition

100 tablets each containing 30 mg of dimethyl 2-{4-[2-(6-benzoyl-2-oxo-1,3-benzoylthiazol-3(2H)-yl)ethoxy]benzyl}malonate and 10 mg of coenzyme Q₁₀

| | | |
|---|---|---|
| Dimethyl 2-{4-[2-(6-benzoyl-2-oxo-1,3-benzoylthiazol-3(2H)- | 3 | g |
| yl)ethoxy]-benzyl}malonate | | |
| Coenzyme Q₁₀ | 1 | g |
| Wheat starch | 20 | g |
| Maize starch | 20 | g |
| Lactose | 30 | g |
| Magnesium stearate | 2 | g |
| Silica | 1 | g |
| Hydroxypropylcellulose | 2 | g |

1-16. (canceled)

17. A composition comprising a combination of a compound favouring lipid and carbohydrate metabolisms and an antioxidant agent.

18. The composition of claim 17, wherein the compound favouring lipid and carbohydrate metabolisms is a compound selected from those of formula (I): 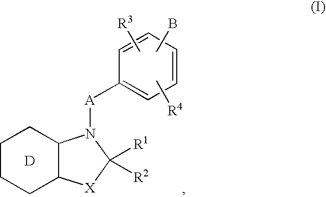
wherein:
X represents oxygen or sulphur or CH₂ or 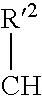
(wherein R¹² together with R² forms an additional bond),
R¹ and R², which may be identical or different, each represents hydrogen, linear or branched (C₁-C₆)alkyl, aryl, aryl-(C₁-C₆)alkyl in which the alkyl moiety may be linear or branched, aryloxy, aryl-(C₁-C₆)alkoxy in which the alkoxy moiety may be linear or branched, linear or branched (C₁-C₆)alkoxy, hydroxy, amino, linear or branched (C₁-C₆)alkylamino or dialkylamino in which the alkyl moieties are linear or branched (C₁-C₆),
or R¹ and R² together form oxo, thioxo or imino, and
R² may also form with R¹² an additional bond,
A represents a (C₁-C₆)alkylene chain in which a CH₂ group may be replaced by a hetero atom selected from oxygen and sulphur, by NRₐ (wherein Rₐ represents hydrogen or linear or branched (C₁-C₆)alkyl), or by phenylene or naphthylene,
B represents a linear or branched (C₁-C₆)alkyl group or a linear or branched (C₂-C₆)alkenyl group, those groups being substituted by an R⁵ group, by a group of formula: 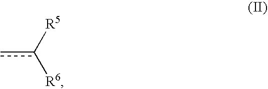
or by a group of formula (III): 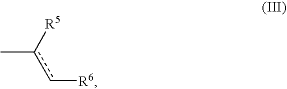
in which groups:
the representation [symbol] denotes that the bond is single or double,
R⁵ represents 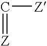
wherein Z represents sulphur or oxygen and Z′ represents OR or NRR′,
and R⁶ represents 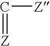
wherein Z″ represents Z′ or R
(wherein R and R′, which may be identical or different, each represents R″ or —C(Me)₂COOR″ wherein R″ represents hydrogen or linear or branched (C₁-C₆)alkyl, linear or branched (C₂-C₆)alkenyl, linear or branched (C₂-C₆)alkynyl, aryl, aryl-(C₁-C₆)alkyl in which the alkyl moiety may be linear or branched, aryl-(C₂-C₆)alkenyl in which the alkenyl moiety may be linear or branched, aryl-(C₂-C₆)alkynyl in which the alkynyl moiety may be linear or branched, heteroaryl, heteroaryl-(C₁-C₆)alkyl in which the alkyl moiety may be linear or branched, heteroaryl-(C₂-C₆)alkenyl in which the alkenyl moiety may be linear or branched, heteroaryl-(C₂-C₆)alkynyl in which the alkynyl moiety may be linear or branched, (C₃-C₈)cycloalkyl, (C₃-C₈)cycloalkyl-(C₁-C₆)alkyl in which the alkyl moiety may be linear or branched, or linear or branched (C₁-C₆)polyhaloalkyl),
R³ and R⁴, which may be identical or different, each represents hydrogen, halogen or R, OR or NRR′ (wherein R and R′ are as defined hereinbefore),
or R³ and R⁴ together with the carbon atoms carrying them, when they are carried by two adjacent carbon atoms, form a ring that has 5 or 6 ring members and that may contain a hetero atom selected from oxygen, sulphur and nitrogen,
D represents:
a benzene nucleus, in which case X may not represent a group 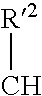
as defined hereinbefore
or D represents a pyridine, pyrazine, pyrimidine or pyridazine nucleus, those five nuclei being unsubstituted or substituted by from 1 to 3 identical or different groups selected from R, OR, S(O)ₙR, C(Z)R, 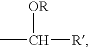
C(Z)OR, NRR′, C(Z)NRR′, 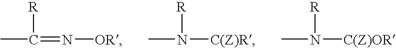
(in which groups R, R′ and Z are as defined hereinbefore and n is 0, 1 or 2), cyano, nitro and halogen atoms,
it being understood that:
when A represents CH₂, B may not represent linear or branched (C₁-C₆)alkyl substituted by 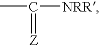
when A and B are in the ortho position in relation to one another on the benzene nucleus carrying them, B may not represent linear or branched (C₂-C₆)-alkenylene substituted by 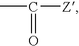
when A represents 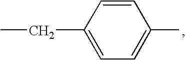
B may not represent —CH₂—COOH
aryl may be a phenyl, naphthyl or biphenyl group, which groups may be partially hydrogenated,
heteroaryl may be any mono- or bi-cyclic aromatic group containing from 5 to 10 ring members, which may be partially hydrogenated on one of the rings in the case of bicyclic heteroaryls, and which contains from 1 to 3 hetero atoms selected from oxygen, nitrogen and sulphur,
wherein the aryl and heteroaryl groups may be substituted by from 1 to 3 groups selected from linear or branched (C₁-C₆)alkyl, linear or branched (C₁-C₆)alkoxy, carboxy, formyl, NR_{b}R_{c} (wherein R_{b} and R_{c}, which may be identical or different, each represents hydrogen, linear or branched (C₁-C₆)alkyl, aryl or heteroaryl), ester, amido, nitro, cyano, O—C(Me)₂COOR″ (wherein R″ is as defined hereinbefore) and halogen,
its enantiomers and diastereoisomers, and addition salts thereof with a pharmaceutically-acceptable acid or base.

19. The composition of claim 17, wherein the compound favouring lipid and carbohydrate metabolisms is dimethyl 2-{4-[2-(6-benzoyl-2-oxo-1,3-benzothiazol-3(2H)-yl)ethoxy]benzyl}malonate, its enantiomers and diastereoisomers, and addition salts thereof with a pharmaceutically-acceptable acid or base.

20. The composition of claim 17, wherein the antioxidant agent is coenzyme Q₁₀.

21. The composition of claim 17, wherein the antioxidant agent is vitamin E.

22. The composition of claim 17, which is a combination of dimethyl 2-{4-[2-(6-benzoyl-2-oxo-1,3-benzothiazol-3(2H)-yl)ethoxy]benzyl}malonate and coenzyme Q₁₀.

23. The composition of claim 17, which is a combination of dimethyl 2-{4-[2-(6-benzoyl-2-oxo-1,3-benzothiazol-3 (2H)-yl)ethoxy]benzyl}malonate and vitamin E.

24. A pharmaceutical composition comprising as active ingredient a composition of claim 17, in combination with one or more pharmaceutically acceptable excipients.

25. A method for treating and/or preventing a living animal body, including a human, afflicted with obesity comprising the step of administering to the living animal body, including a human, an amount of a composition of claim 17 which is effective for alleviation of obesity.

26. A method for treating and/or preventing a living animal body, including a human afflicted with an overweight condition characterised by a body mass index greater than 25 and less than 30 comprising the step of administering to the living animal body, including a human, an amount of a composition of claim 17 which is effective for alleviation of the condition.

27. A method for treating and/or preventing a living animal body, including a human, afflicted with obesity caused by a therapeutic treatment, comprising the step of administering to the living animal body, including a human, an amount of a composition of claim 17 which is effective for alleviation of obesity caused by a therapeutic treatment.

28. A method for treating and/or preventing a living animal body, including a human, afflicted with obesity caused by treatment for type I or II diabites, comprising the step of administering to the living animal body, including a human, an amount of a composition of claim 17 which is effective for alleviation of obesity caused by treatment for type I or II diabetes.

29. A method for treating and/or preventing a living animal body, including a human, afflicted with an overweight condition characterised by a body mass index greater than 25 and less than 30 caused by a therapeutic treatment, comprising the step of administering to the living animal body, including a human, an amount of a composition of claim 17 which is effective for alleviation of the condition.

30. A method for treating and/or preventing a living animal body, including a human, afflicted with an overweight condition characterised by a body mass index greater than 25 and less than 30 caused by treatment for type I or II diabetes comprising the step of administering to the living animal body, including a human, an amount of a composition of claim 17 which is effective for alleviation of the condition.
